(19) United States
(12) Patent Application Publication
(10) Pub. No.: US 2026/0165618 A1
(43) Pub. Date: Jun. 18, 2026

(54) WORKING ELECTRODE FOR A CONTINUOUS BIOLOGICAL MONITOR

(71) Applicant: Allez Health Inc., Carlsbad, CA (US)

(72) Inventor: Robert James Boock, Carlsbad, CA (US)

(73) Assignee: Allez Health Inc., Carlsbad, CA (US)

(21) Appl. No.: 19/397,332

(22) Filed: Nov. 21, 2025

Related U.S. Application Data
(60) Provisional application No. 63/734,223, filed on Dec. 16, 2024.

(51) Int. Cl. A61B 5/1468; A61B 5/145

(52) U.S. Cl. CPC A61B 5/1468; A61B 5/14532; A61B 2562/125

(57) ABSTRACT

A method for fabricating a working electrode for a continuous biological monitor is disclosed. The method includes providing a substrate. A compound is prepared from an aqueous solution of a conductive carbon material, boronic acid and an electrophore, and an elastomeric material. The compound is applied to the substrate, and the compound is cured on the substrate to form a cured compound. A working electrode for a continuous biological monitor is disclosed. The working electrode includes a substrate and a cured compound disposed on the substrate. The cured compound includes a conductive carbon material, boronic acid and an electrophore, and an elastomeric material.

### RELATED APPLICATIONS

This application claims priority to U.S. Provisional Patent Application No. 63/734,223, filed on Dec. 16, 2024, and entitled “Working Electrode for a Continuous Biological Sensor with Boronic Acid”; the contents of which are incorporated by reference in full.

### BACKGROUND

Medical patients often have diseases or conditions that require the measurement and reporting of biological conditions. For example, if a patient has diabetes, it is important that the patient have an accurate understanding of the level of glucose in their blood. Traditionally, diabetes patients have monitored their glucose levels by sticking their finger with a small lance, allowing a drop of blood to form, and then dipping a test strip into the blood. The test strip is positioned in a handheld monitor that performs an analysis on the blood and visually reports the measured glucose level to the patient. Based upon this reported level, the patient makes important decisions on what food to consume, or how much insulin to inject into their blood. Although it would be advantageous for the patient to check glucose levels many times throughout the day, many patients fail to adequately monitor their glucose levels due to the pain and inconvenience. As a result, the patient may eat improperly or inject either too much or too little insulin. Either way, the patient has a reduced quality of life and increased chance of doing permanent damage to their health and body. Diabetes is a devastating disease that if not properly controlled can lead to terrible physiological conditions such as kidney failure, skin ulcers, or bleeding in the eyes, and eventually blindness, pain and the eventual amputation of limbs.

Regular and accurate monitoring of glucose levels is critical for diabetes patients. To facilitate such monitoring, continuous glucose monitoring (CGM) sensors are a type of device in which glucose is automatically measured from fluid sampled in an area just under the skin multiple times a day. CGM devices typically involve a small housing in which the electronics are located and which is adhered to the patient's skin to be worn for a period of time. A small needle within the device delivers the subcutaneous sensor which is often electrochemical. In this way, a patient may install a CGM on their body, and the CGM will provide automated and accurate glucose monitoring for many days without any action required from the patient or a caregiver. It will be understood that depending upon the patient's needs, that continuous glucose monitoring may be performed at different intervals. For example, some continuous glucose monitors may be set or programmed to take multiple readings per minute, whereas in other cases the continuous glucose monitor can be programmed or set to take readings every hour or so. It will be understood that a continuous glucose monitor may sense and report readings at different intervals.

Continuous glucose monitoring is a complicated process, and it is known that glucose levels in the blood can significantly rise/increase or lower/decrease quickly, due to several causes. Accordingly, a single glucose measurement provides only a snapshot of the instantaneous level of glucose in a patient's body. Such a single measurement provides little information about how the patient's use of glucose is changing over time, or how the patient reacts to specific dosages of insulin. Accordingly, even a patient that is adhering to a strict schedule of strip testing will likely be making incorrect decisions as to diet, exercise, and insulin injection. Of course, this is exacerbated by a patient that is less consistent on performing their strip testing. To give the patient a more complete understanding of their diabetic condition and to get a better therapeutic result, some diabetic patients are now using continuous glucose monitoring.

Electrochemical glucose sensors operate by using electrodes which typically detect an amperometric signal caused by oxidation of enzymes during conversion of glucose to gluconolactone. The amperometric signal can then be correlated to a glucose concentration. Two-electrode (also referred to as two-pole) designs use a working electrode and a reference electrode, where the reference electrode provides a reference against which the working electrode is biased. The reference electrodes essentially complete the electron flow in the electrochemical circuit. Three-electrode (or three-pole) designs have a working electrode, a reference electrode and a counter electrode. The counter electrode replenishes ionic loss at the reference electrode and is part of an ionic circuit.

Conventional CGM systems typically use a working wire that uses a core of tantalum on which a thin layer of platinum is deposited. Tantalum is a relatively stiff material, so is able to be pressed into the skin without bending, although an introducer needle may be used to facilitate insertion. Further, it is inexpensive as compared to platinum, which makes for an economical working wire. As is well known, an enzyme layer is deposited over the platinum layer, which is able to accept oxygen molecules and glucose molecules from the user's blood. The key chemical processes for glucose detection occur within the enzyme membrane. Typically, the enzyme membrane has one or more glucose oxidase enzymes (GOx) dispersed within the enzyme membrane. When a molecule of glucose and a molecule of oxygen (O₂) are combined in the presence of the glucose oxidase, a molecule of gluconate and a molecule of hydrogen peroxide (H₂O₂) are formed. In one construction, the platinum surface facilitates a reaction wherein the hydrogen peroxide reacts to produce water and hydrogen ions, and two electrons are generated. The electrons are drawn into the platinum by a bias voltage placed across the platinum wire and a reference electrode. In this way, the magnitude of the electrical current flowing in the platinum is intended to be related to the number of hydrogen peroxide reactions, which is intended to be related to the number of glucose molecules oxidized. A measurement of the electrical current on the platinum wire can thereby be associated with a particular level of glucose in the patient's blood or interstitial fluid (ISF).

Unfortunately, the current cost of using a continuous glucose monitor is prohibitive for many patients that could benefit greatly from its use. As described generally above, a continuous glucose monitor has two main components. First, there is a housing for the electronics, processor, memory, wireless communication, and power. The housing is typically reusable, and reusable over extended periods of time, such as months. This housing then connects or communicates to a disposable CGM sensor that is adhered to the patient's body, which typically uses an introducer needle to subcutaneously insert the sensor into the patient. This sensor must be replaced, sometimes as often as every three days, and likely at least once every other week. Thus, the cost to purchase new disposable sensors represents a significant financial burden to patients and insurance companies. Because of this, a substantial number of patients that could benefit from continuous glucose monitoring are not able to use such systems and are forced to rely on the less reliable and painful finger stick monitoring.

Further, having direct contact between the enzyme layer and the platinum layer has other disadvantages. First, the actual useful exposed area of an exposed portion of the platinum wire is substantially reduced by oxidation contamination, which also may lead to unpredictable and undesirable sensitivity results. In order to overcome this deficiency, the sensor must be subjected to sophisticated and on-going calibration. Further, the bias voltage between the platinum wire and the reference electrode must be set relatively high, for example between 0.4-1.0 V. Such a high bias voltage is required to draw the electrons into the platinum wire, but also acts to attract contaminants from the blood or ISF into the sensor. These contaminants such as acetaminophen and uric acid interfere with the chemical reactions, leading to false and misleading glucose level readings.

The working wire is then associated with a reference electrode, and in some cases one or more counter electrodes, which form the CGM sensor. In operation, the CGM sensor is coupled to and cooperates with electronics in a small housing in which, for example, a processor, memory, a wireless radio, and a power supply are located. The CGM sensor typically has a disposable applicator device that uses a small introducer needle to deliver the CGM sensor subcutaneously into the patient. Once the CGM sensor is in place, the applicator is discarded, and the electronics housing is attached to the sensor. Although the electronics housing is reusable and may be used for extended periods, the CGM sensor and applicator need to be replaced quite often, usually every few days. In such known CGM sensors, the electronics housing has all the supporting electronics for the sensor in the sensor housing, such as an analog front end, processor, memory, and radio, as well as the battery. Typically, the battery will have some trickle-power sensing circuit that can detect when the electronics housing is coupled to the CGM sensor. Once such a detection is sensed, then the battery can be used to fully power the electronics and the working wire in the CGM sensor. In this way, the battery must be sized to allow for low-power sensing for extended periods of time, which can extend for a year or more, and have sufficient reserve power to operate the CGM sensors that it detects. As the electronics housing is reusable on multiple CGM sensors, the battery must be sized to handle the expected number of uses.

CGMs are crucial for managing conditions like diabetes by providing real-time glucose monitoring. These systems typically use a working electrode and a reference electrode to detect analyte concentrations through electrochemical reactions. Enzyme-based sensors, which commonly employ enzymes like glucose oxidase to catalyze glucose reactions, have limitations including enzyme instability, signal drift, and interference from other compounds, reducing sensor accuracy and longevity. These issues have prompted the exploration of alternative sensing chemistries to improve the reliability and performance of CGM devices.

Known in the art are compositions that use boronic acid-based synthetic redox-active receptors, which electrochemically sense target analytes by forming reversible complexes with diols, such as glucose. These compositions can be coated onto a variety of conductive electrodes, including rhodium, platinum, gold, carbon nanotubes, and graphene. For example, redox-active compositions may involve boronic acid-functionalized polymers, such as electropolymerized aniline or pyrrole derivatives, that change redox potential in response to the target analyte. Some systems also use impedance-based detection, or hydrogel matrices blended with boronic acid polymers, to improve sensitivity and biocompatibility. Despite these advancements, challenges remain in achieving stable, reproducible, and biocompatible sensors that can operate effectively in-vivo over extended periods.

### SUMMARY

Disclosed herein is a method for fabricating a working electrode for a continuous biological monitor. The method includes providing a substrate. A compound is prepared that is an aqueous solution of a conductive carbon material, boronic acid and an electrophore, and an elastomeric material. The compound is applied to the substrate, and the compound is cured to form a cured compound.

Disclosed herein is a working electrode for a continuous biological monitor. The working electrode includes a substrate and a cured compound disposed on the substrate. The cured compound includes a conductive carbon material, boronic acid and an electrophore, and an elastomeric material.

### BRIEF DESCRIPTION OF THE DRAWINGS

FIG. 1 is a not-to-scale cross-sectional diagram of a sensor for a continuous biological monitor, in accordance with some aspects.

FIG. 2 depicts an example of a wire-based 3-pole system of a sensor for a continuous biological monitor, in accordance with some aspects.

FIG. 3 is a schematic of the formulation of an aqueous solution for a compound comprising boronic acid, in accordance with some aspects.

FIG. 4 is a flowchart for fabricating a working electrode for a continuous biological monitor, in accordance with some aspects.

### DETAILED DESCRIPTION

Glucose sensing technology has traditionally relied on enzyme-based detection systems, which are prone to stability and performance issues over time. As an alternative, boronic acid has been explored as a synthetic, non-enzymatic receptor due to its ability to selectively and reversibly bind with saccharides like glucose. When boronic acid reacts with glucose, it forms a reversible covalent bond with the hydroxyl groups on the sugar, resulting in the formation of a cyclic boronate ester. This reaction is particularly advantageous for glucose sensing because of the selective and reversible binding nature of boronic acid with glucose.

Efforts known in the art to leverage boronic acid for glucose sensing have led to the development of optical and fluorescence-based sensors. In these systems, boronic acid is typically conjugated to optical dyes or fluorophores, forming a complex that changes its emission wavelength in response to glucose binding. This method, however, presents challenges, as the reaction is often non-linear, typically following a third-order or fourth-order polynomial response. These sensors are generally built using fiber-optic waveguides or similar structures, which incorporate LED emitters and photodetectors. However, the optical components required for such systems result in larger and bulky electronics, making the sensors durable but not easily or practically disposable.

Boronic acid-based fluorescent sensors are often encapsulated within hydrogels to accommodate the structural rearrangements required during glucose binding. However, hydrogels present mechanical challenges, particularly in maintaining attachment to substrates with sufficient strength for in-vivo use. Directly attaching a hydrogel to an optical fiber can lead to mechanical failure in biological environments, necessitating the development of support structures or tie-layers to improve stability.

Despite their oxygen independence and minimal interference from other sugars, boronic acid-based optical sensors face additional challenges, such as photo-bleaching and performance degradation over multiple excitation cycles. Consequently, these systems are often operated in a pulsed format to extend their in-vivo lifespan. However, their equilibrium-based nature makes them less effective in situations where rapid glucose concentration changes occur, and their performance in hyperglycemic conditions is limited due to inherent non-linearity, which is influenced by the glucose-limiting membrane.

Systems and methods disclosed herein provide an innovative working electrode that integrates boronic acid functionality into a conductive material such as carbon, enhancing accuracy, stability, and durability in continuous biological monitoring applications. The conductive material or conductor, such as a carbon-based material, can be chosen from a diverse range of options, allowing for enhanced flexibility in both design and functionality. An innovative aspect of this approach lies in embedding the boronic-based molecule (e.g., boronic acid with an electrophore), directly within the conductor itself. By positioning the molecule internally into a working electrode, this method allows for more efficient electron transfer and stronger molecular interactions, leading to enhanced conductivity and stability within the system. This unique configuration represents a significant advancement over conventional designs, where the molecule is typically applied on the surface or in proximity to the conductor rather than integrated within it. Another innovative aspect involves the integration of an electrophore with the boronic acid, which enhances the electron-transfer characteristics of the system and significantly improves the boronic acid's reactivity with glucose.

Attaching boronic acid to an electrophore is complex and not straightforward. The methods and systems described herein position the boronic acid in close proximity to the electrophore, where a direct covalent bond may be used but is not required. Furthermore, the components of the sensing materials themselves form a conductive medium. In conventional systems, charge conduction occurs along a polymer chain to the working electrode. In contrast, in the present approach boronic acid is integrated into a fully conductive medium, effectively making it part of the working electrode. By placing the electrophore near the boronic acid within this conductive matrix, charge transfer occurs through the medium itself.

In the present disclosure, the enhanced responsiveness of boronic acid moieties integrated into conductive carbon, compared to conventional boronic acid sensing approaches, enables amperometric electrochemical sensing. Amperometric electrochemistry also benefits from well-established, scalable manufacturing techniques and simpler electronic components, making it cost-effective and suitable for mass production. The structural simplicity and robustness of non-enzymatic sensors, which do not rely on biological units, contribute to improved quality control and consistent performance under varying storage and use conditions. An amperometric system with boronic acid known in the art has been attempted, but with boronic acid as a separate layer on a conductive working electrode.

Conventional boronic acid amperometric systems often fail in practical applications due to poor charge conduction. This inefficiency arises because the charge transfer distance between the boronic acid and conductor is too great to achieve a significant signal amplification. By incorporating the boronic acid with the electrophore directly into the conductor (e.g., carbon material) in the present disclosure, the charge transfer path is significantly shortened. This configuration allows for intimate contact between the boronic acid and the carbon material, eliminating the need for charge transfer along a polymer chain or through an encapsulation membrane.

Boronic acid has the capability to bind with a variety of diols beyond glucose, including dopamine and alpha hydroxy acids such as lactic acid. These analytes, among others, are highly relevant for applications in body-worn sensors. In some cases, the sensor incorporating boronic acid with the electrophore directly into the carbon material can detect dopamine levels, making it a valuable tool for monitoring mood and mental health conditions, such as depression. Boronic acid groups have a strong affinity for catechol structures, like those found in dopamine, enabling selective and sensitive detection. By measuring fluctuations in dopamine concentration, this sensor can provide real-time insights into a person's neurochemical state and overall well-being. Such technology may aid in assessing mood variations, identifying patterns related to depressive episodes, and potentially guiding personalized treatments or interventions for mental health.

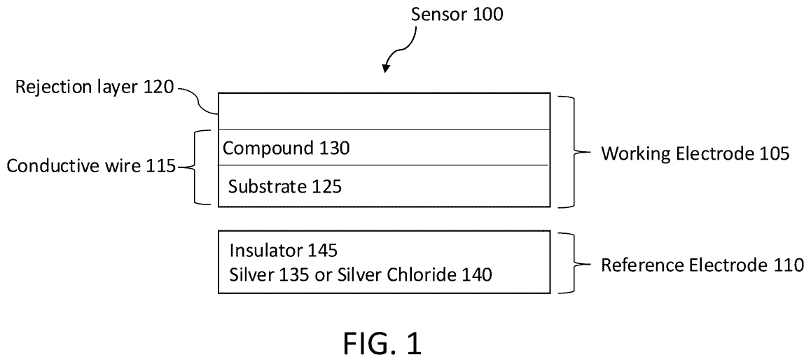

FIG. 1 is a not-to-scale cross-sectional diagram of a sensor for a continuous biological monitor, in accordance with some aspects. A sensor 100 for a continuous biological monitor has a working electrode 105 (disposed on a substrate 125, e.g., a working wire) which cooperates with a reference electrode 110 to provide an electrochemical reaction that can be used to detect analyte concentrations through electrochemical reactions in a patient's blood or interstitial fluid (ISF). Although sensor 100 is illustrated with one working electrode 105 and one reference electrode 110, it will be understood that in some cases, sensors may use multiple working electrodes, multiple reference electrodes, and counter electrodes. It will also be understood that sensor 100 may have different physical relationships between the working electrode 105 and the reference electrode 110. For example, the working electrode 105 and the reference electrode 110 may be arranged in layers, spiraled, arranged concentrically, or side-by-side. It will be understood that many other physical arrangements may be consistent with the disclosure herein.

The sensor 100 has a reference electrode 110 may be separate from the working electrode 105. In this way, the manufacture of the working electrode 105 is simplified and can be performed with a consistency that contributes to dramatically improved stability and performance. The reference electrode 110 provides a reference against which the working electrode 105 is biased, and essentially completes the electron flow in the electrochemical circuit.

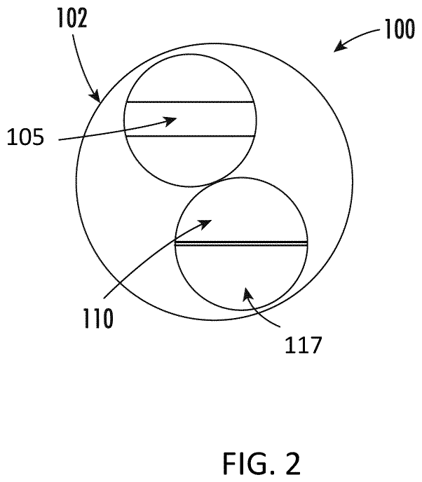

In some cases, the sensor 100 may be a wire-based 2-pole system where the reference electrode 110 may be an elongated wire with a circular cross-section. In some cases, the sensor 100 may be a wire-based 3-pole system. FIG. 2 depicts an example of a wire-based 3-pole system of a sensor for a continuous biological monitor, in accordance with some aspects. In this example, a split-wire configuration is implemented which enables three wires of the working electrode 105, reference electrode 110 and a counter electrode 117 to occupy the space of only two full wires. For example, the reference electrode 110 and the counter electrode 117 have a semi-circular cross-section such that the two wires coupled together occupy the space of one full wire while providing nearly the same surface area as two full wire electrodes. Therefore, the 3-pole design can fit within the inner diameter of a typical insertion needle 102 instead of three full wires which would require a larger insertion needle.

Referring to FIG. 1, the reference electrode 110 may be composed of silver 135 or silver chloride 140 and may be coated with an insulator 145. This insulator can function as an ion-limiting layer that is non-conductive to electrons.

The working electrode 105 may be an elongated wire having a circular cross-section. It will be understood that the working wire may have other cross-sectional shapes such as square, rectangular, triangular, or other geometric shapes. It will further be understood that the working wire may take other forms, such as a plate or ribbon.

The working electrode 105 has a conductive portion, which is shown for sensor 100 in FIG. 1 as a conductive wire 115. The conductive wire 115 has a conductive surface (i.e., conducting layer) that allows the flow of electric current across its surface. It possesses electrical conductivity, meaning it contains free-moving charges, such as electrons or ions, that enable the conduction of electricity. The substrate 125 of conductive wire 115 may be comprised of, for example, plastic, metal, composite, or a combination thereof. In some examples, the substrate 125 of conductive wire 115 may be comprised of plastic, carbon, platinum, a platinum coating on a less expensive metal, among others. It will be understood that other electron conductors may be used consistent with this disclosure.

In the example of FIG. 1, the working electrode 105 includes a substrate 125 which is then coated with a compound 130. Put another way, a cured compound 130 is disposed on the substrate 125. The substrate 125 may be comprised of a plastic material and the material may be selected from polyethylene, polypropylene, polystyrene, polyvinyl chloride, or polylactic acid. In some cases, the substrate 125 can be a fibrous material, such as paper or wood. These options may be appealing in specific markets as a cost-effective alternative. In other cases, a substrate 125 comprised of a metal such as tantalum may be used.

The compound 130 may be formulated as a coating from an aqueous solution and subsequently applied to the substrate 125. An aqueous solution refers to a liquid mixture where water is the solvent, and various compounds or reagents are dissolved in it to facilitate the electrode's preparation or modification.

In some examples, the working electrode 105 for sensor 100 may utilize a rejection layer 120 applied over the conductive wire 115, or over the cured compound 130 and/or substrate 125. The rejection layer 120 is a solid coating that protects the boronic acid and may selectively block or filter out unwanted substances or “interferents” from reaching the electrode surface. This layer allows only specific target molecules to pass through while excluding other compounds that could produce false readings or noise. The rejection layer 120 may function as a combination or hybrid of a biocompatible protective layer and an interference layer. This layer serves to eliminate substances produced by the body, such as hydrogen peroxide that can deactivate boronic acid, as well as other interferent species. In some cases, this is achieved through charge-based selectivity, where the layer's inherent or alternating charge repels oppositely charged molecules, and through molecular size exclusion. By leveraging an alternating charge mechanism, the rejection layer 120 dynamically controls interactions with incoming molecules, improving the specificity and accuracy of the sensor's readings by minimizing electrochemical noise and interference.

The rejection layer 120 has an inherent charge (positive or negative) or alternates charges depending on the external environment. This charge prevents oppositely charged interferents, such as ions or molecules that could alter sensor readings, from passing through the layer. For example, negatively charged molecules would be repelled by a negatively charged rejection layer.

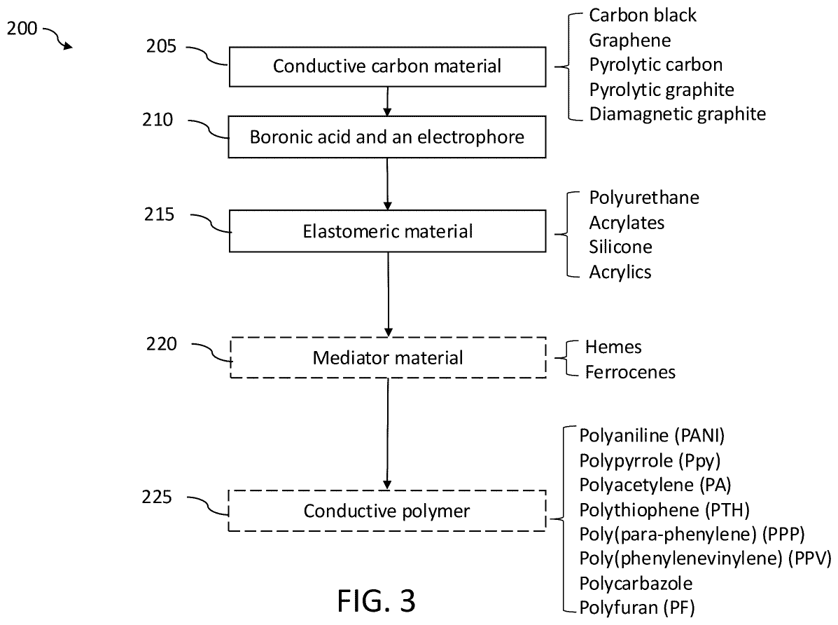

FIG. 3 is a schematic of the formulation of an aqueous solution for a compound, in accordance with some aspects. In some cases, an aqueous solution 200 (e.g., compound 130) includes a conductive carbon material 205, boronic acid and an electrophore 210, and an elastomeric material 215. The carbon material 205 in the aqueous solution 200 is selected from a group comprising one or more of carbon black, pyrolytic graphite, graphene, pyrolytic carbon, and diamagnetic graphite or combinations thereof. In some cases, the carbon material 205 comprises carbon black, pyrolytic graphite and graphene.

To enhance conductivity in carbon-based systems, incorporating pyrolytic graphite is particularly effective. While carbon black alone may be used, achieving adequate conductivity necessitates very high loading levels, which often results in elevated resistivity. By blending pyrolytic graphite with carbon black, the overall resistivity of the system is significantly reduced. For example, a composition of 70% carbon black and 30% pyrolytic graphite may denote a higher concentration of pyrolytic graphite, while a mixture of 60% carbon black combined with 20% pyrolytic graphite and 20% graphene offers further optimization. In further examples, other percentage compositions are possible. This approach allows for precise tuning of the resistivity, aiming to achieve a resistivity of the compound of less than 100 ohms per square millimeter (Ω/mm²), such as below 50 Ω/mm² while having a high conductivity. Lowering resistivity in this manner reduces background signals and minimizes current losses, thereby improving the signal-to-noise ratio and ensuring that the detected current predominantly reflects the desired signal. This optimization results in improved accuracy and performance for devices utilizing such systems.

The aqueous solution 200 also includes boronic acid with an electrophore 210. Boronic acid is an organic compound containing a boron atom connected to two hydroxyl groups and an alkyl or aryl group. It has the general formula R—B(OH)₂, where R is the alkyl or aryl group. When a boronic acid molecule has an electrophore, it refers to the addition of an electron-withdrawing group to the boronic acid structure, which increases the reactivity of the boronic acid. The electrophore is a substituent or functional group that attracts electrons, and the presence of the electrophore on the boronic acid enhances its ability to participate in various chemical reactions by increasing the acidity of the boron atom and making the boronic acid more electrophilic. In sensor applications, the reactivity of boronic acid toward specific analytes, such as diols or sugars, can be adjusted by incorporating electron-withdrawing groups.

An electrophore facilitates charge conduction, meaning it enables the transfer of charge from one material to another. It acts as an electron shuttle without altering the properties of the materials involved. An electrophore is a component that enables electrons to move through a medium that is either inherently conductive or remains chemically unchanged during the transfer process. In some examples, the electrophore comprises a 1) bipyridinium alkyne; 2) short aminomethyl or tertiary amine spacers (e.g., in polyallylamine); 3) phenylene or direct aryl links (e.g., on polyaniline, graphene, or PEDOT); 4) 3-acrylamidophenylboronic acid (APBA or mAPBA) which provides meta-phenylene-amide linker widely used in poly(N-isopropylacrylamide) (PNIPAAm); or 5) 4-vinylphenylboronic acid (or its pinacol ester-protected form) which may provide a p-phenylene linker reacted on the boronic acid.

An electrophore compound, such as a bipyridinium alkyne is linked to boronic acid compound. Linked may be any type of attachment such as a covalent bond, hydrogen bond, or a charge attachment system. The electrophore compound is electrochemically reduced in the context of sensing analytes and can accept electrons. For example, it can undergo electrochemical reduction during analyte sensing and may function as an electron acceptor. Electron transfer results in a measurable change in current by a potentiostat, with the magnitude of the current being proportional to the analyte concentration. However, these compounds may have difficulty in measuring glucose in fixed layer membrane systems, as the distance from active electrochemical reaction site (such as in platinum or carbon) can impede accurate sensing.

The aqueous solution 200 also includes an elastomeric material 215. The elastomeric material 215 may be chosen for its mechanical properties, such as strength and flexibility. Elastomeric materials are necessary for maintaining structural integrity because stronger elastomeric materials allow for higher loading content of the boronic acid in the compound. Elastomeric materials are inherently electrically insulative, so incorporating them into a conductive medium is non-obvious and counterintuitive. In the disclosed approach, the elastomer serves as a structural binder while conductive loading surrounds it, enabling electrical continuity. This configuration allows the elastomer to act as an adhesive or mechanical binder that holds the system together without compromising overall conductivity. Achieving high loading is important to minimizing the resistivity of the conductive wire 115. Various elastomeric materials may be used to achieve the desired characteristics, including polyurethane, acrylate, silicone, or acrylic. For example, the elastomeric material may comprise a polyurethane, acrylate, silicone, or acrylic. It is understood that other suitable elastomeric materials may also be substituted as needed. In some cases, the elastomeric material may be self-crosslinking, for example, a self-crosslinking acrylate. This refers to an acrylate-based polymer that contains functional groups within its structure that can react with each other, forming crosslinks without the need for an external crosslinking agent. When these polymers are subjected to appropriate conditions—such as exposure to heat, light, or specific chemical triggers—the functional groups on the polymer chains react, resulting in a three-dimensional, crosslinked network. This method imparts desirable properties to the material, such as improved durability, chemical resistance, and mechanical strength.

In one case, the method of making the compound 130 involves an aqueous solution 200 of dispersion of a polymer that contains a trace amount of a solvent. As the drying or evaporation process begins, the water content evaporates first, leading to an increased concentration of the remaining solvent within the solution. This gradual concentration increase creates a solvent-rich microenvironment surrounding the polymer particles. The localized solvent enrichment enables the polymer particles to soften or partially melt, allowing them to merge and begin forming a continuous film or membrane. As drying continues, the remaining solvent and water gradually evaporate, causing the polymer particles to further coalesce and solidify into a cohesive, durable membrane structure. In some examples, this may be a liquid, gel or paste. This approach ensures that the film exhibits enhanced uniformity and mechanical integrity due to the efficient merging of the polymer particles and controlled evaporation process, eliminating the need for additional crosslinking agents.

In some cases, the aqueous solution 200 (e.g., compound 130), optionally further comprises a mediator 220. The incorporation of mediators 220 may enhance the electron transfer process, ensuring reliable and efficient electrochemical performance. In some examples, the mediator 220 comprises hemes, such as iron-containing molecules, or ferrocenes. Ferrocenes are a class of organometallic compound consisting of a sandwich-like structure where an iron (Fe) atom is “sandwiched” between two rings of cyclopentadienyl (C₅H₅) anions.

In some cases, the aqueous solution 200 (e.g., compound 130), optionally, further comprises a conductive polymer 225 for electron transfer enhancement. These may be referred to as electron transfer enhancers. The conductive polymer 225 comprises at least one of polyaniline (PANI), polypyrrole (PPy), polyacetylene (PA), polythiophene (PTH), poly(para-phenylene) (PPP), poly(phenylenevinylene) (PPV), polycarbazole, and polyfuran (PF). By incorporating the conductive polymer 225, the resistivity of the compound 130 can be adjusted to a desired level.

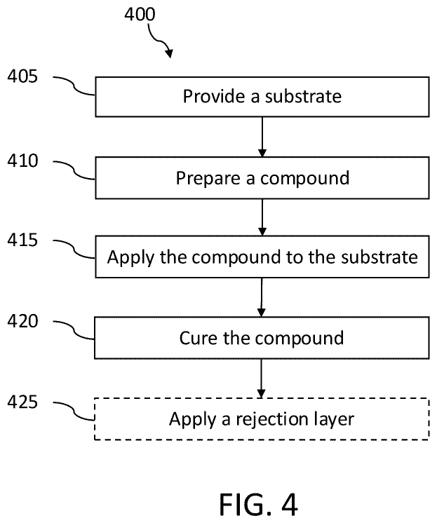

FIG. 4 is a flowchart for a method for fabricating a working electrode for a continuous biological monitor, in accordance with some aspects. The particular steps, order of steps, and combination of steps are shown for illustrative and explanatory purposes only. Other aspects can implement different particular steps, orders of steps, and combinations of steps to achieve similar functions or results. A method 400 for fabricating a working electrode starts at step 405, where a substrate 125 is provided. At step 410, a compound 130 is formulated from an aqueous solution 200 which may optionally include a mediator 220 and/or a conductive polymer 225. At step 415, the compound 130 is applied to the substrate 125. This may be applied or deposited on substrate 125 using any suitable technique, including dip coating, extrusion, spin coating, screen printing, and similar methods. In some cases, the compound 130 is a conductive paste when applied to the substrate 125 (e.g., by extrusion on a wire, or screen printing on a sheet/plate/ribbon substrate) and then cured on the substrate. In other cases, the compound 130 may be applied as a liquid (e.g., by dip coating). At step 420, the substrate 125 with the applied compound 130 is cured. Encapsulating the boronic acid in the compound 130 of the working electrode 105 allows electron transfer to occur without any intervening distance between the boronic acid molecules and the conductive material, creating direct and intimate contact with the conductive carbon materials 205 (e.g., conductor). This configuration significantly enhances conductivity and optimizes charge transfer efficiency, enabling the boronic acid electrochemistry and amperometric system to operate effectively.

In one example, the compound 130 may be comprised of the formulation of (% by weight for a total of 100%):
50-80% of carbon material
20-50% of boronic acid
1-2% of elastomeric material
1-2% of mediator and/or conductive polymer (optional)
0-10% additional water

In step 420, the compound 130 is cured to form a cured compound 130. The curing method may use a stepwise reduction in humidity to optimize the structural arrangement and conductivity of the conductor material. Put another way, the curing may include step reduction in humidity of an oven over a period of time. Initially, the material is placed in a humidified oven environment, gradually transitioning to a pure dry oven. This controlled approach deliberately slows down the drying process, allowing the material to rearrange optimally and settle into its lowest potential energy state, which corresponds to the highest conductivity. Unlike conventional flash curing, where immediate drying can yield suboptimal conductivity, this method provides sufficient time for the material to achieve an arrangement with minimal resistivity. By carefully managing humidity levels during the curing method, enhanced electrical performance of the material is achieved.

For example, the curing method may start at 90% humidity for 5 minutes, followed by a reduction to 80% humidity for 3 minutes, then 65% humidity for 3 minutes, and continue decreasing in controlled steps until reaching a completely dry environment. In another example, the humidity is reduced by 10% to 20% every 2-8 minutes. This gradual reduction in humidity ensures that the drying process is slow and allows the material to rearrange optimally, achieving the desired high-conductivity state.

At step 425, the rejection layer 120 (e.g., biocompatible layer) is applied on the cured compound 130. The rejection layer compound may be non-conductive, ion-permeable, and selectively permeable based on molecular weight. Additionally, it can be electrodeposited or dip-coated as a thin, conformal layer to ensure precise and uniform coverage. It will be appreciated that other methods to apply the rejection layer 120 may be used.

In some cases, for example, the rejection layer 120 is designed to address and minimize the impact of secondary sugars that may interact with the boronic acid. Unlike traditional concerns over interfering species such as acetaminophen, the primary challenge involves larger secondary sugars, such as dextrose and maltose, which can be present in trace amounts and have the potential to generate false signals. The rejection layer 120 selectively blocks or mitigates the effects of these sugars, enhancing the sensor's accuracy and reliability. In this context, the rejection layer 120 protects the boronic acid as opposed to purely rejecting electrochemical species. It will be understood that additional layers may be applied over the rejection layer 120 based on the specific biological target being tested and the requirements of the particular application.

Traditional electrochemical systems based on boronic acid typically require coulombometric measurements, which are impractical for battery-powered devices due to high power demands. In contrast, non-enzymatic, boronic acid-based amperometric detection offers benefits. Amperometric measurements quantify the electric current (amperes) at a fixed potential, while coulometric measurements quantify the total electric charge (coulombs) passed over a period of time, typically during an exhaustive reaction. This method uses a redox-driven, equilibrium-based process that is both reversible and free of byproducts, providing enhanced stability and longer sensor lifetimes.

Systems and methods disclosed utilize the advantages of an amperometric measurement approach by positioning the boronic acid 210 in close proximity to the carbon material 205, enabling precise and efficient measurements. This configuration allows operation at significantly lower bias voltages (approximately 0.0-0.2 V compared to about 0.55 V for enzyme-based systems) and improves charge conduction by embedding boronic acid directly within the conductive carbon material. Lower voltage minimizes interference and background current, while the shortened electron-transfer path enables rapid and robust signal generation without the exhaustive charge integration required for coulometric systems.

In contrast, conventional systems using boronic acid often rely on coulombic measurements, which require complex and costly electronics that are difficult to miniaturize. Amperometric measurement systems, on the other hand, offer benefits in terms of simplicity, cost-efficiency, and ease of circuit fabrication. Pulsed amperometry can further enhance performance by refreshing the electrode surface between pulses and improving equilibrium dynamics for reversible boronic acid-diol binding, resulting in faster response and extended sensor life. This distinction is unique, as coulombic systems typically involve larger, bulkier electronics, resulting in less practical and more cumbersome devices. By adopting an amperometric system, the development of smaller, more practical, and wearable sensors becomes possible, broadening potential applications while ensuring a cost-effective and user-friendly solution.

Boronic acid-based electrochemistry significantly reduces potential interference, particularly from compounds like acetaminophen, by operating at much lower bias voltages such as 0.0 to 0.2 volts compared to the standard 0.55 volts for conventional amperometric systems. This lower voltage range minimizes background signals, thereby enhancing the specificity and accuracy of the glucose detection process. Additionally, using pulsed amperometric signal electronics rather than continuous amperometric electronics can enhance the equilibrium reaction dynamics. Traditionally, the calibration equation is expressed as:
y=(m*x)+b; where y is a displayed value, m is a sensitivity, x is a measured current and b is a constant background.

Because the optimized electrode design and low bias voltage nearly eliminate background current, the constant background b in the calibration equation is no longer needed. This applies to both pulsed and non-pulsed amperometric methods, allowing the equation to simplify to:
y=(m*x); where y is a displayed value, m is a sensitivity and x is a measured current.

This improvement ensures consistent sensitivity from bench testing to in-vivo environments and reduces complexity in calibration. This overcomes the difficulties faced by enzyme-based, factory-calibrated sensors, which often struggle to align in-vitro sensitivity measurements with in-vivo performance.

### CLAUSES

Clause 1. A method for fabricating a working electrode for a continuous biological monitor comprising: providing a substrate; preparing a compound from an aqueous solution of a conductive carbon material, boronic acid and an electrophore, and an elastomeric material; applying the compound to the substrate; and curing the compound on the substrate to form a cured compound.

Clause 2. The method of clause 1, wherein the conductive carbon material is selected from a group comprising one or more of carbon black, pyrolytic graphite, graphene, pyrolytic carbon, and diamagnetic graphite.

Clause 3. The method of any of clauses 1-2, wherein the boronic acid is linked to the electrophore with a covalent bond, a hydrogen bond, or a charge attachment.

Clause 4. The method of any of clauses 1-3, wherein the electrophore comprises a bipyridinium alkyne.

Clause 5. The method of any of clauses 1-4, wherein the elastomeric material comprises a polyurethane, acrylate, silicone, or acrylic.

Clause 6. The method of any of clauses 1-5, wherein the compound further comprises a mediator, the mediator comprising hemes or ferrocenes.

Clause 7. The method of any of clauses 1-6, wherein the compound further comprises a conductive polymer comprising at least one of polyaniline (PANI), polypyrrole (PPy), polyacetylene (PA), polythiophene (PTH), poly(para-phenylene) (PPP), poly(phenylenevinylene) (PPV), polycarbazole, and polyfuran (PF).

Clause 8. The method of any of clauses 1-7, wherein the curing comprises a step reduction in humidity of an oven over a period of time.

Clause 9. The method of any of clauses 1-8, further comprising applying a rejection layer on the cured compound.

Clause 10. The method of any of clauses 1-9, wherein the compound has a resistivity less than 100 ohms per square millimeter (Ω/mm²).

Clause 11. A working electrode for a continuous biological monitor comprising: a substrate; a cured compound disposed on the substrate, the cured compound comprising: a conductive carbon material; boronic acid and an electrophore; and an elastomeric material.

Clause 12. The working electrode of clause 11, wherein the conductive carbon material is selected from a group comprising one or more of carbon black, pyrolytic graphite, graphene, pyrolytic carbon, and diamagnetic graphite.

Clause 13. The working electrode of clause 11, wherein the conductive carbon material comprises carbon black, pyrolytic graphite and graphene.

Clause 14. The working electrode of any of clauses 11-13, wherein the electrophore comprises a bipyridinium alkyne.

Clause 15. The working electrode of any of clauses 11-14, wherein the elastomeric material comprises a polyurethane, acrylate, silicone, or acrylic.

Clause 16. The working electrode of any of clauses 11-15, wherein the compound further comprises a mediator, the mediator comprising hemes or ferrocenes.

Clause 17. The working electrode of any of clauses 11-16, wherein the compound further comprises a conductive polymer comprising at least one of polyaniline (PANI), polypyrrole (PPy), polyacetylene (PA), polythiophene (PTH), poly(para-phenylene) (PPP), poly(phenylenevinylene) (PPV), polycarbazole, and polyfuran (PF).

Clause 18. The working electrode of any of clauses 11-17, further comprising applying a rejection layer on the cured compound.

Clause 19. The working electrode of any of clauses 11-18, wherein the compound has a resistivity less than 100 ohms per square millimeter (Ω/mm²).

Clause 20. The working electrode of any of clauses 11-19, wherein the cured compound is formed from an aqueous solution.

Reference has been made in detail to aspects of the disclosed invention, one or more examples of which have been illustrated in the accompanying figures. Each example has been provided by way of explanation of the present technology, not as a limitation of the present technology. In fact, while the specification has been described in detail with respect to specific aspects of the invention, it will be appreciated that those skilled in the art, upon attaining an understanding of the foregoing, may readily conceive of alterations to, variations of, and equivalents to these examples. For instance, features illustrated or described as part of one example may be used with another example to yield a still further example. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only, and is not intended to limit the invention.

What is claimed is:

1. A method for fabricating a working electrode for a continuous biological monitor, comprising:
providing a substrate;
preparing a compound from an aqueous solution of a conductive carbon material, boronic acid and an electrophore, and an elastomeric material;
applying the compound to the substrate; and
curing the compound on the substrate to form a cured compound.

2. The method of claim 1, wherein the conductive carbon material is selected from a group comprising one or more of carbon black, pyrolytic graphite, graphene, pyrolytic carbon, and diamagnetic graphite.

3. The method of claim 1, wherein the boronic acid is linked to the electrophore with a covalent bond, a hydrogen bond, or a charge attachment.

4. The method of claim 1, wherein the electrophore comprises a bipyridinium alkyne.

5. The method of claim 1, wherein the elastomeric material comprises a polyurethane, acrylate, silicone, or acrylic.

6. The method of claim 1, wherein the compound further comprises a mediator, the mediator comprising hemes or ferrocenes.

7. The method of claim 1, wherein the compound further comprises a conductive polymer comprising at least one of polyaniline (PANI), polypyrrole (PPy), polyacetylene (PA), polythiophene (PTH), poly(para-phenylene) (PPP), poly(phenylenevinylene) (PPV), polycarbazole, and polyfuran (PF).

8. The method of claim 1, wherein the curing comprises a step reduction in humidity of an oven over a period of time.

9. The method of claim 1, further comprising:
applying a rejection layer on the cured compound.

10. The method of claim 1, wherein the compound has a resistivity less than 100 ohms per square millimeter (Ω/mm²).

11. A working electrode for a continuous biological monitor, comprising:
a substrate; and
a cured compound disposed on the substrate, the cured compound comprising:
a conductive carbon material;
boronic acid and an electrophore; and
an elastomeric material.

12. The working electrode of claim 11, wherein the conductive carbon material is selected from a group comprising one or more of carbon black, pyrolytic graphite, graphene, pyrolytic carbon, and diamagnetic graphite.

13. The working electrode of claim 11, wherein the conductive carbon material comprises carbon black, pyrolytic graphite and graphene.

14. The working electrode of claim 11, wherein the electrophore comprises a bipyridinium alkyne.

15. The working electrode of claim 11, wherein the elastomeric material comprises a polyurethane, acrylate, silicone, or acrylic.

16. The working electrode of claim 11, wherein the cured compound further comprises a mediator, the mediator comprising hemes or ferrocenes.

17. The working electrode of claim 11, wherein the cured compound further comprises a conductive polymer comprising at least one of polyaniline (PANI), polypyrrole (PPy), polyacetylene (PA), polythiophene (PTH), poly(para-phenylene) (PPP), poly(phenylenevinylene) (PPV), polycarbazole, and polyfuran (PF).

18. The working electrode of claim 11, further comprising:
a rejection layer disposed on the cured compound.

19. The working electrode of claim 11, wherein the cured compound has a resistivity less than 100 ohms per square millimeter (Ω/mm²).

20. The working electrode of claim 11, wherein the cured compound is formed from an aqueous solution.
